(19) United States
(12) Patent Application Publication
(10) Pub. No.: US 2024/0257668 A1
(43) Pub. Date: Aug. 1, 2024

(54) 3D ARTIFICIAL SKIN MODEL AND METHOD FOR PRODUCING THE SAME

(71) Applicant: Korea Advanced Institute of Science and Technology, Daejeon (KR)

(72) Inventors: Pilnam Kim, Daejeon (KR); Youngmin Jo, Daejeon (KR)

(21) Appl. No.: 18/423,327

(22) Filed: Jan. 26, 2024

(30) Foreign Application Priority Data
Jan. 27, 2023 (KR) 10-2023-0010756

(51) Int. Cl. G09B 23/30; C12N 5/071; G01N 33/50

(52) U.S. Cl. CPC G09B 23/306; C12N 5/0602; C12N 5/0625; G01N 33/5082; C12N 2501/15; C12N 2501/72; C12N 2501/90; C12N 2501/998

(57) ABSTRACT

The present disclosure relates to a three-dimensional (3D) artificial skin model, and specifically, to a 3D artificial skin model that perfectly mimics the ridge shape of the dermal-epidermal junction, and a method for producing the same. Normal skin tissue has wavy ridges formed at the dermal-epidermal junction (DEJ), but the wavy ridges are known to flatten with aging. The artificial skin model of the present disclosure is a 3D artificial skin model that perfectly mimics the shape of the ridges at the dermal-epidermal junction. Since the artificial skin model mimics the structure and function of normal skin, it is expected to be widely used in the pharmaceutical and cosmetic fields.

### BACKGROUND

### 1. Technical Field

The present disclosure relates to a three-dimensional (3D) artificial skin model, and specifically, to a 3D artificial skin model that perfectly mimics the shape of the ridges at the dermal-epidermal junction, and a method for producing the same.

### 2. Related Art

The skin is the largest organ in the human body and serves as a barrier to protect the body. The skin is structurally divided into two tissue layers: epidermis and dermis. The epidermis is an epithelial tissue exposed to the outside and serves to protect the inside of the skin and prevent evaporation of water, and the dermis is a thick cell layer beneath the epidermis and is involved in the elasticity, gloss, and tension of the skin. The basement membrane lies between the epidermis and the dermis. Normal skin tissue has wavy ridges formed at the dermal-epidermal junction (DEJ), but the wavy ridges are known to flatten with aging.

Meanwhile, in modern times, with the development of industrial society and the increase in social activities, the self-consciousness of wanting to actively express oneself is increasing, and interest in beauty and suppressing skin aging is increasing. At the same time, as awareness of animal welfare increases, the need for alternative animal testing methods that can replace animal testing in the development of drugs or cosmetics is emerging. Therefore, there is an urgent need for artificial skin models for use in skin aging research and drug development. These artificial skin models should be able to mimic the structure and function of normal skin, but there is still no artificial skin model that can mimic the wavy ridges of the dermal-epidermal junction in normal skin tissue.

Therefore, the present disclosure has been made in order to solve the above-described problems, and relates to a 3D artificial skin model that perfectly mimics the shape of the ridges at the dermal-epidermal junction, and a method for producing the same. The artificial skin model of the present disclosure, which significantly overcomes the problems of the prior art, is expected to be widely used in the pharmaceutical and cosmetic fields.

### SUMMARY

One object of the present disclosure is to provide a multilayer-structured artificial skin.

Another object of the present disclosure is to provide a method for producing a multilayer-structured artificial skin.

Still another object of the present disclosure is to provide a method of screening a candidate substance for inhibiting skin aging using a multilayer-structured artificial skin.

Yet another object of the present disclosure is to provide a method of evaluating the effect of a substance for inhibiting skin aging using a multilayer-structured artificial skin.

Still yet another object of the present disclosure is to provide a method of studying skin aging using a multilayer-structured artificial skin.

However, objects to be achieved by the present disclosure are not limited to the objects mentioned above, and other objects not mentioned above may be clearly understood by those skilled in the art from the following description.

Hereinafter, various embodiments described herein will be described with reference to figures. In the following description, numerous specific details are set forth, such as specific configurations, compositions, and processes, etc., in order to provide a thorough understanding of the present disclosure. However, certain embodiments may be practiced without one or more of these specific details, or in combination with other known methods and configurations. In other instances, known processes and preparation techniques have not been described in particular detail in order to not unnecessarily obscure the present disclosure. Reference throughout this specification to “one embodiment” or “an embodiment” means that a particular feature, configuration, composition, or characteristic described in connection with the embodiment is included in at least one embodiment of the present disclosure. Thus, the appearances of the phrase “in one embodiment” or “an embodiment” in various places throughout this specification are not necessarily referring to the same embodiment of the present disclosure. Additionally, the particular features, configurations, compositions, or characteristics may be combined in any suitable manner in one or more embodiments.

Unless otherwise stated in the specification, all the scientific and technical terms used in the specification have the same meanings as commonly understood by those skilled in the technical field to which the present disclosure pertains.

Throughout the specification, it is to be understood that when any part is referred to as “including” or “containing” any component, it does not exclude other components, but may further include other components, unless otherwise specified.

The human skin is composed of a multilayer structure and is roughly divided into an epithelial layer (epidermis) containing epithelial cells, a basement membrane, and a stromal layer (dermis) containing stromal cells. The multilayer structure of the skin has a special double wrinkle structure, which undergoes various changes depending on age, disease, etc. Outer wrinkles, which are seen externally, are commonly referred to as skin wrinkles, and become deeper and larger as aging progresses. Inner wrinkles form at the dermal-epidermal junction (DEJ) starting from the fetal period (see FIG. 1), and, contrary to outer wrinkles, tend to flatten as aging progresses (see FIG. 2).

In modern times, with the development of industrial society and the increase in social activities, the self-consciousness of wanting to actively express oneself is increasing, and interest in beauty and suppressing skin aging is increasing. At the same time, as awareness of animal welfare increases, the need for alternative animal testing methods that can replace animal testing in the development of drugs or cosmetics is emerging. Therefore, there is an urgent need for artificial skin models for use in skin aging research and drug development. However, conventionally developed artificial skin models have a limitation in that they cannot mimic the inner wrinkles of the dermal-epidermal junction.

In one aspect of the present disclosure, the present disclosure provides a multilayer-structured artificial skin including: a first layer including collagen and stromal cells; and a second layer including cellulose acetate and collagen.

The first layer including collagen and stromal cells corresponds to the dermal layer of skin tissue, and is hereinafter referred to as “stromal layer” in the present disclosure.

The second layer including cellulose acetate and collagen corresponds to the basement membrane existing between the epithelial layer and the dermal layer in skin tissue, and is hereinafter referred to as “basement membrane” in the present disclosure.

In addition to the first layer and the second layer, the artificial skin of the present disclosure may further include a third layer including epithelial cells.

The third layer including epithelial cells corresponds to the epithelial layer of skin tissue, and is hereinafter referred to as “epithelial layer” in the present disclosure.

In the artificial skin of the present disclosure, the second layer is located on the first layer, and the third layer is located on the second layer.

The stromal cells included in the first layer are cells that constitute fibrous connective tissue, and may be any one or more types of cells selected from the group consisting of fibroblasts, chondroblasts, osteoblasts, neuroglial cells, adipocytes, macrophages, and plasma cells. Particularly, the stromal cells may be any one or more type of cells selected from the group consisting of osteoblasts, osteocytes, and osteoclasts.

The epithelial cells included in the third layer are cells that form a layer of cells and constitute a membrane tissue that covers the inside and outside of a tissue or gland, and these epithelial cells may be any one or more types of cells selected from the group consisting of simple squamous epithelium, simple cuboidal epithelium, simple columnar epithelium, stratified squamous epithelium, stratified cuboidal epithelium, stratified columnar epithelium, pseudostratified epithelium, transitional epithelium, and glandular epithelium cells.

In the artificial skin of the present disclosure, the first layer and the second layer are characterized in that they are attached to each other by a bioadhesive, and the bioadhesive is preferably transglutaminase 2 (TR2), without being limited thereto.

The artificial skin of the present disclosure is characterized in that ridges are formed at the junction between the first layer and the second layer. The junction between the first layer and the second layer corresponds to the dermal-epidermal junction in normal skin tissue, and the ridges herein correspond to the inner wrinkles that form at the dermal-epidermal junction in normal skin tissue starting from the fetal period. Also, the ridges herein have a wavy shape. It may be understood that, in the wavy shape, as the distance between one ridge and another ridge adjacent thereto is shorter, the density of the ridges is higher, which may mean that wrinkles are dense or the number of wrinkles is large.

The artificial skin of the present disclosure may be incubated in a medium containing transforming growth factor-beta 2 (TGF-β2), thereby completing the formation of ridges at the junction between the first layer and the second layer. Specifically, the medium may contain serum or serum albumin, without being limited thereto.

In another aspect of the present disclosure, the present disclosure provides a method for producing a multilayer-structured artificial skin, the method including steps of: preparing a first layer including collagen and stromal cells; and preparing a second layer including cellulose acetate and collagen.

The first layer including collagen and stromal cells corresponds to the dermal layer of skin tissue, and is hereinafter referred to as “stromal layer” in the present disclosure.

The second layer including cellulose acetate and collagen corresponds to the basement membrane existing between the epithelial layer and the dermal layer in skin tissue, and is hereinafter referred to as “basement membrane” in the present disclosure.

The method for producing a multilayer-structured artificial skin according to the present disclosure may further include preparing a third layer including epithelial cells, in addition to preparing the first layer and preparing the second layer.

The third layer including epithelial cells corresponds to the epithelial layer of skin tissue, and is hereinafter referred to as “epithelial layer” in the present disclosure.

In the method for producing an artificial skin according to the present disclosure, the second layer is located on the first layer, and the third layer is located on the second layer.

The stromal cells included in the first layer are cells that constitute fibrous connective tissue, and may be any one or more types of cells selected from the group consisting of fibroblasts, chondroblasts, osteoblasts, neuroglial cells, adipocytes, macrophages, and plasma cells. Particularly, the stromal cells may be any one or more types of cells selected from the group consisting of osteoblasts, osteocytes, and osteoclasts.

The epithelial cells included in the third layer are cells that form a layer of cells and constitute a membrane tissue that covers the inside and outside of a tissue or gland, and these epithelial cells may be any one or more types of cells selected from the group consisting of simple squamous epithelium, simple cuboidal epithelium, simple columnar epithelium, stratified squamous epithelium, stratified cuboidal epithelium, stratified columnar epithelium, pseudostratified epithelium, transitional epithelium, and glandular epithelium cells.

In the method for producing an artificial skin according to the present disclosure, the first layer and the second layer are characterized in that they are attached to each other by a bioadhesive, and the bioadhesive is preferably transglutaminase 2 (TR2), without being limited thereto.

The artificial skin produced by the method for producing an artificial skin according to the present disclosure is characterized in that ridges are formed at the junction between the first layer and the second layer. The junction between the first layer and the second layer corresponds to the dermal-epidermal junction in normal skin tissue, and the ridges herein correspond to the inner wrinkles that form at the dermal-epidermal junction in normal skin tissue starting from the fetal period. Also, the ridges herein have a wavy shape. It may be understood that, in the wavy shape, as the distance between one ridge and another ridge adjacent thereto is shorter, the density of the ridges is higher, which may mean that wrinkles are dense or the number of wrinkles is large.

The method for producing an artificial skin according to the present disclosure may further include incubating the first layer and the second layer in a medium containing transforming growth factor-beta 2 (TGF-β2). The artificial skin produced by the method for producing an artificial skin according to the present disclosure may be incubated in a medium containing TGF-β2, thereby completing the formation of ridges at the junction between the first layer and the second layer. Specifically, the medium may contain serum or serum albumin, without being limited thereto.

In still another aspect of the present disclosure, the present disclosure provides a method for screening a candidate substance for inhibiting skin aging, the method including steps of: preparing the artificial skin according to the one aspect of the present disclosure; treating the artificial skin with a candidate substance for inhibiting skin aging; and checking changes in ridges at the junction between the first and second layers in the artificial skin.

As used herein, “screening” refers to selecting a substance with any desired property from a candidate group consisting of various substances by a specific testing or evaluation method.

The artificial skin used in the screening method according to the present disclosure is characterized in that ridges are formed at the junction between the first layer and the second layer. The junction between the first layer and the second layer corresponds to the dermal-epidermal junction in normal skin tissue, and the ridges herein correspond to the inner wrinkles that form at the dermal-epidermal junction in normal skin tissue starting from the fetal period. Also, the ridges herein have a wavy shape. It may be understood that, in the wavy shape, as the distance between one ridge and another ridge adjacent thereto is shorter, the density of the ridges is higher, which may mean that wrinkles are dense or the number of wrinkles is large. Therefore, in the screening method, if the density of the ridges in the artificial skin after treatment with the candidate substance for inhibiting skin aging increased compared to that before treatment with the candidate substance, the candidate substance may be determined to be effective in inhibiting skin aging. On the other hand, if the density of the ridges in the artificial skin after treatment with a candidate substance for accelerating skin aging instead of the candidate substance for inhibiting skin aging decreased compared to that before treatment with the candidate substance for accelerating skin aging, this may be interpreted to have the same meaning as “the inner wrinkles that form at the dermal-epidermal junction in normal skin tissue starting from the fetal period flatten with aging”. Thus, the candidate substance for accelerating skin aging may be determined to be effective in accelerating skin aging.

By controlling the density of the ridges through this method, research on the mechanism of skin aging is possible.

Additionally, by checking changes in the density of the ridges through this method, it is possible to develop a pharmaceutical composition, food composition, or cosmetic composition for inhibiting or ameliorating skin aging. The active ingredient of the pharmaceutical composition, food composition, or cosmetic composition may be the candidate substance for inhibiting skin aging selected through the screening method of the present disclosure, without being limited thereto.

In this case, for the purposes of the present disclosure, the pharmaceutical composition is for inhibiting or ameliorating skin aging, and may further be for preventing or treating various skin diseases caused by skin aging. The above pharmaceutical composition contains a candidate substance for inhibiting skin aging as an active ingredient, and may further contain a protein and a pharmaceutically acceptable carrier, excipient or diluent, which are involved therein. The “pharmaceutically acceptable” carrier or excipient means one approved by a regulatory agency of the Federal or a state government, or one listed in the governmental pharmacopoeia or other generally recognized pharmacopoeia for use in vertebral animals, and more particularly in humans. Considering that the pharmaceutical composition of the present disclosure is applied to skin tissue, for parenteral administration, the pharmaceutical composition may be in the form of suspensions, solutions, or emulsions, in oily or aqueous vehicles, and may be in the form of solid or semi-solid. Furthermore, the pharmaceutical composition of the present disclosure may contain formulatory agents such as suspending, stabilizing, solubilizing, and/or dispersing agents, and may be sterilized. The pharmaceutical composition can be stable under the conditions of manufacture and storage and can be preserved against the contaminating action of microorganisms such as bacteria and fungi. Alternatively, the pharmaceutical composition of the present disclosure may be in sterile powder form for reconstitution with a suitable vehicle before use. The pharmaceutical composition may be presented in unit dose form, in micro-needle patches, in ampoules, or other unit-dose containers, or in multi-dose containers. Alternatively, the pharmaceutical composition can be stored in a freeze-dried (lyophilized) condition requiring only the addition of sterile liquid carrier, for example, water for injection immediately prior to use. Extemporaneous injection solutions and suspensions may be prepared from sterile powders, granules or tablets. In certain non-limiting embodiments, the pharmaceutical composition of the present disclosure may be formulated as liquid or contained as microspheres in liquids. In certain non-limiting embodiments, the pharmaceutical composition of the present disclosure may contain pharmaceutically acceptable compounds and/or mixtures thereof at concentrations between 0.001 and 100,000 U/kg. In addition, in certain non-limiting embodiments, excipients that are suitable for the pharmaceutical composition of the present disclosure may include preservatives, suspending agents, stabilizers, dyes, buffers, antibacterial agents, antifungal agents, and isotonic agents, for example, sugars or sodium chloride. As used herein, the term “stabilizer” refers to a compound optionally used in the pharmaceutical composition of the present disclosure in order to increase storage life. In non-limiting embodiments, stabilizers may be sugars, amino acids or polymers. In addition, the pharmaceutical composition of the present disclosure may contain one or more pharmaceutically acceptable carriers. The carrier may be a solvent or dispersion medium. Non-limiting examples of pharmaceutically acceptable carriers include water, saline, ethanol, polyols (e.g., glycerol, propylene glycol and liquid polyethylene glycol), oils, and suitable mixtures thereof. Non-limiting examples of sterilization techniques that are applied to the pharmaceutical composition of the present disclosure include filtration through a bacterial-retaining filter, terminal sterilization, incorporation of sterilizing agents, irradiation, sterilizing gas irradiation, heating, vacuum drying, and freeze drying.

In addition, for the purposes of the present disclosure, the food composition may be for inhibiting or ameliorating skin aging, and specifically may be one having skin antioxidant, whitening, anti-inflammatory and anti-wrinkle activities. As used herein, “antioxidant” refers to inhibiting oxidation, thereby inhibiting cellular aging. Oxygen that enters the human body through breathing creates energy necessary for the human body while creating free radicals, which are harmful excess oxygen. Since free radicals attack normal cells in the body, causing aging or various diseases, removing free radicals is a way to prevent oxidation (aging) of cells, and suppressing this cellular oxidation is referred to as anti-oxidation. As used herein, “whitening” refers to a function of preventing melanin pigment from being excessively deposited on the skin due to increased melanocytes caused by continuous exposure of the skin to ultraviolet rays, or a function of thinning previously deposited melanin pigment. Accordingly, the food composition can suppress the formation of spots, freckles, age spots, and melanoma caused by excessive melanin pigmentation. As used herein, “anti-inflammatory” refers to the effect of alleviating and resisting inflammation. Anti-inflammation in the skin can be considered to act to protect, stabilize, disinfect and sterilize the skin. As used herein, “wrinkles” is meant to encompass outer wrinkles, which are seen externally, and inner wrinkles formed at the dermal-epidermal junction. In addition, “anti-wrinkle” means delaying, inhibiting or ameliorating wrinkle formation.

The food composition of the present disclosure, which contains a candidate substance for inhibiting skin aging selected through the screening method of the present disclosure, may be prepared in the form of various foods, for example, beverages, gums, teas, vitamin complexes, powders, granules, tablets, capsules, confectionery, cakes, bread, and the like. Since the food composition of the present disclosure is a composition with little or no toxicity and side effects, it may be used with confidence even when it is taken for a long period of time for preventive purposes. When the composition of the present disclosure is contained in the food composition, it may be added in an amount of 0.1 to 100 wt % based on the total weight. When the food composition is prepared in the form of a beverage, there is no particular limitation, except that the beverage contains the food composition at the indicated percentage. The beverage may additionally contain various flavorings or natural carbohydrates, like conventional beverages. Examples of the natural carbohydrates include monosaccharides such as glucose, disaccharides such as fructose, polysaccharides such as sucrose, conventional sugars such as dextrin, cyclodextrin, and the like, and sugar alcohols such as xylitol, sorbitol, erythritol, and the like. Examples of the flavorings include natural flavorings (thaumatin, stevia extracts, such as rebaudioside A, glycyrrhizin, etc.) and synthetic flavorings (saccharin, aspartame, etc.). In addition, the food composition of the present disclosure may contain various nutrients, vitamins, minerals (electrolytes), flavorings such as synthetic flavorings and natural flavorings, colorants, pectic acid and salts thereof, alginic acid and salts thereof, organic acids, protective colloidal thickeners, pH adjusting agents, stabilizers, preservatives, glycerin, alcohol, carbonizing agents that are used in carbonated beverages, etc. These components may be used individually or in combination. Although the percentage of such additives is not of great importance, it is generally selected in a range of 0.1 to about 100 parts by weight based on 100 parts by weight of the composition of the present disclosure.

In addition, for the purposes of the present disclosure, the cosmetic composition in the present disclosure may be for inhibiting or ameliorating skin aging, and specifically may be one having skin antioxidant, whitening, anti-inflammatory and anti-wrinkle activities. The cosmetic composition of the present disclosure, which contains a candidate substance for inhibiting skin aging selected through the screening method of the present disclosure, may be prepared in the form of skin softener, nutritional lotion, nutritional essence, massage cream, cosmetic bath additive, body lotion, body milk, bath oil, baby oil, baby powder, shower gel, shower cream, sunscreen lotion, sunscreen cream, suntan cream, skin lotion, skin cream, sunscreen cosmetics, cleansing milk, depilatory (for cosmetics), face and body lotion, face and body cream, skin whitening cream, hand lotion, hair lotion, cosmetic cream, jasmine oil, bath soap, liquid soap, cosmetic soap, shampoo, hand sanitizer (hand cleaner), medicated soap (non-medical use), cream soap, facial wash, hair rinse, beauty soap, teeth whitening gel, toothpaste, etc. To this end, the composition of the present disclosure may further contain a suitable carrier, excipient or diluent that is commonly used in the preparation of cosmetic compositions. Examples of the carrier, excipient or diluent that may be further added to the cosmetic composition of the present disclosure include, but are not limited to, purified water, oils, wax, fatty acids, fatty acid alcohols, fatty acid esters, surfactants, humectants, thickeners, antioxidants, viscosity stabilizers, chelating agents, buffers, lower alcohols, and the like. In addition, the cosmetic composition may, if necessary, contain whitening agents, moisturizers, vitamins, sunscreens, perfumes, dyes, antibiotics, antibacterial agents, and antifungal agents. As the oils described above, hydrogenated vegetable oil, castor oil, cottonseed oil, olive oil, palm oil, jojoba oil, and/or avocado oil may be used, and as the wax, beeswax, mellow, carnauba, candelilla, montan, ceresin, liquid paraffin, and/or lanolin may be used. As the fatty acid, stearic acid, linoleic acid, linolenic acid and/or oleic acid may be used, and as the fatty acid alcohol, cetyl alcohol, octyl dodecanol, oleyl alcohol, panthenol, lanolin alcohol, stearyl alcohol, and/or hexadecanol may be used. As the fatty acid ester, isopropyl myristate, isopropyl palmitate and/or butyl stearate may be used. Surfactants that may be used in the cosmetic composition include cationic surfactants, anionic surfactants and nonionic surfactants known in the art, while surfactants derived from natural products are preferably used. In addition, the cosmetic composition may contain a humectant, a thickener, an antioxidant, etc., which are widely known in the cosmetic field and whose types and amounts are as known in the art.

Hereinafter, the present disclosure will be described in detail with reference to examples.

The artificial skin model of the present disclosure is a 3D artificial skin model that perfectly mimics the shape of the ridges at the dermal-epidermal junction. Since the artificial skin model may mimic the structure and function of normal skin, it is expected to be widely used in the pharmaceutical and cosmetic fields.

### BRIEF DESCRIPTION OF THE DRAWINGS

FIG. 1 is a schematic diagram showing the structure of normal skin tissue.

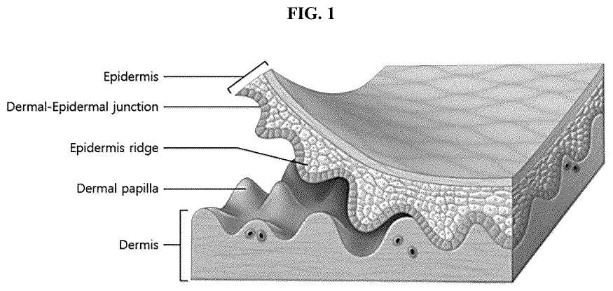

FIG. 2 is a schematic diagram showing aging-induced structural changes in the dermal-epidermal junction in normal skin tissue.

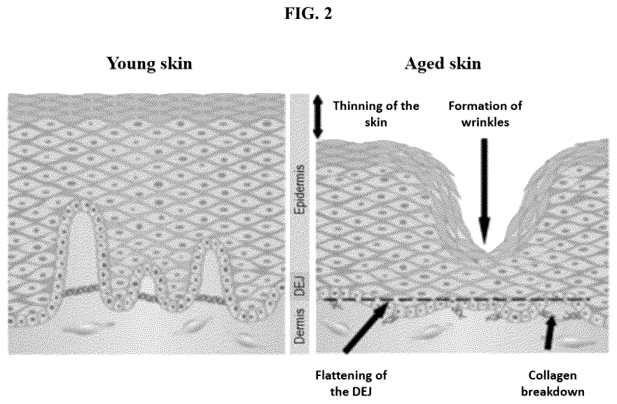

FIG. 3 is a schematic diagram showing steps of producing a three-dimensional (3D) artificial skin model according to one embodiment of the present disclosure.

FIG. 4 shows the overall structure of the three-dimensional (3D) artificial skin model produced according to one embodiment of the present disclosure.

FIG. 5 shows the results of evaluating the effect of TGF-β2 on tissue contraction at the stromal layer when incubating the three-dimensional (3D) artificial skin model, produced according to one embodiment of the present disclosure, in a medium containing TGF-β2. In FIG. 5, the scale bar is 6 mm.

FIG. 6 shows the results of quantifying the effect of TGF-β2 on tissue contraction at the stromal layer when incubating the three-dimensional (3D) artificial skin model, produced according to one embodiment of the present disclosure, in a medium containing TGF-β2.

FIG. 7 shows the results of evaluating the effect of blebbistatin on the inhibition of tissue contraction at the stromal layer when incubating the three-dimensional (3D) artificial skin model, produced according to one embodiment of the present disclosure, in a medium containing blebbistatin. In FIG. 7, the scale bar is 200 μm.

FIG. 8 is a result of confirming that the three-dimensional (3D) artificial skin model produced according to one embodiment of the present disclosure well mimics the shape of the ridges at the dermal-epidermal junction. In FIG. 7, the scale bar is 1000 μm.

FIG. 9 shows the results of testing the importance of the presence of basement membrane (CVM) in the three-dimensional (3D) artificial skin model produced according to one embodiment of the present disclosure. In FIG. 9, the scale bar is 200 μm.

FIG. 10 shows the results of testing the importance of coating of the basement membrane with TR2 in the three-dimensional (3D) artificial skin model produced according to one embodiment of the present disclosure. In FIG. 10, the scale bar is 400 μm.

### DETAILED DESCRIPTION

Hereinafter, the present disclosure will be described in detail with reference to the following examples. However, the following examples are merely illustrative of the present disclosure, and the content of the present disclosure is not limited by the following examples.

### EXAMPLES

### Example 1. Production of 3D Artificial Skin Model

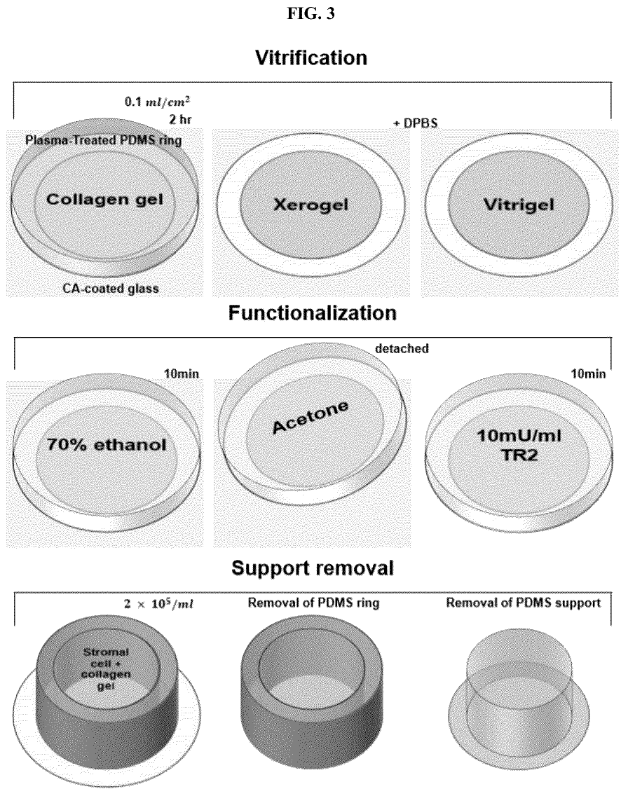

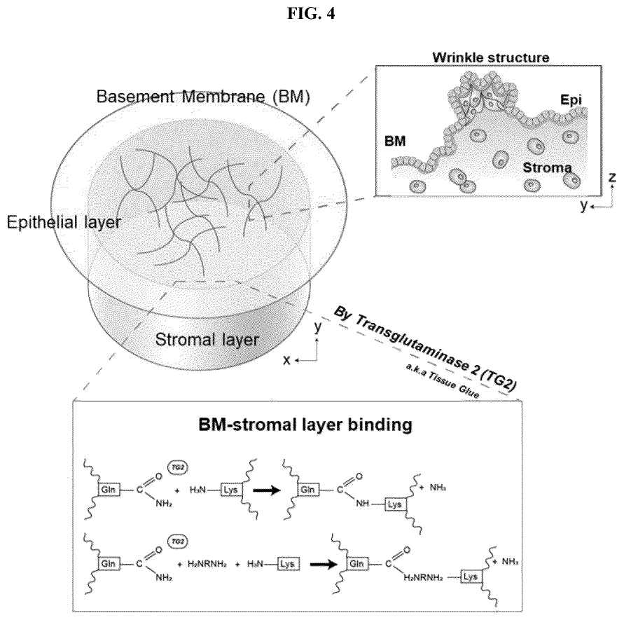

A three-dimensional (3D) artificial skin model of the present disclosure was produced through the steps of vitrification, functionalization, and support removal. FIGS. 3 and 4 show a schematic diagram of steps of producing the 3D artificial skin model and the overall structure of the 3D artificial skin model, respectively. Hereinafter, the production method will be descried in detail.

### Example 1-1. Preparation of Basement Membrane Premix

A basement membrane premix was prepared by mixing 0.25 mg/ml of type I collagen, 2.3% (relative to type I collagen) of 1M NaOH, 10% (relative to total volume) 10×PBS, 10% (relative to total volume) of serum-free DMEM (Dulbecco's Modified Eagle Medium), 0.05% (relative to type I collagen) of Col-F collagen binding reagent, and distilled water.

### Example 1-2. Preparation of Stromal Layer Premix

A stromal layer premix was prepared by mixing 2 mg/ml of type I collagen, 2.3% (relative to type I collagen) of 1M NaOH, 10% (relative to total volume) of 10×PBS, and DMEM containing 1×10⁵/ml to 2×10⁵/ml of stromal cells.

### Example 1-3. Production of Basement Membrane Model

10 mg/ml of cellulose acetate (CA) solution was placed on a round glass plate and spin-coated at 1000 rpm for 60 seconds to form a sacrificial layer having a thickness of 110 nm. A donut-shaped polydimethylsiloxane (PDMS) ring with a thickness of 300 μm, an inner diameter of 8 mm and an outer diameter of 12 mm was treated with oxygen plasma and placed on the CA-coated glass plate, and 122 μl of the basement membrane premix of Example 1-1 was poured into each PDMS ring and incubated at 37° C. for 2 hours. The resulting collagen gel was then vitrified in a refrigerator at 50% humidity and 18° C. temperature for one week. The completely vitrified basement membrane was rehydrated in DPBS for 10 min and sterilized with 70% ethanol for 10 min. Thereafter, the basement membrane on the glass plate was transferred to a dish containing acetone. In this step, as the CA sacrificial layer was dissolved immediately, the basement membrane could be easily harvested. The harvested basement membrane was immersed again in 70% ethanol to remove acetone, and then rehydrated by immersion in DPBS.

### Example 1-4. Production of Basement Membrane+Stromal Layer Model

The basement membrane of Example 1-3 was placed on a flat parafilm, and Matrigel diluted in DPBS at a ratio of 1/50 was added thereto and incubated at 37° C. for 30 minutes, thereby coating the basement membrane with Matrigel. Next, the basement membrane was further coated by treatment with 10 mU/ml TR2 (transglutaminase 2 in serum-free DMEM) as a bioadhesive at room temperature for 10 minutes. Excess TR2 was removed by pipetting, and a hollow cylindrical PDMS support coated with Pluronic F-127 (0.5% in 70% EtOH) was inserted into the inner hole of the donut-shaped basement membrane ring, and 85 μl of the stromal layer premix of Example 1-2 was added to the cylindrical support. Next, the resulting structure was incubated in an incubator for 2 hours for gelation and adhesion. Thereafter, the gel support was removed, and DMEM was poured to separate the basement membrane+stromal layer tissue from the parafilm surface. The separated tissue was turned over, thus producing a basement membrane+stromal layer model with the stromal layer at the bottom and the basement membrane at the top.

### Example 1-5. Production of Epithelial Layer+Basement Membrane+Stromal Layer Model

Epithelial cells were seeded at 1×10⁵/well in the basement membrane+stromal layer tissue of Example 1-4 and adhered by incubation at 37° C. for 15 minutes. After confirming adhesion, the entire tissue was transferred to a dish filled with DMEM (supplemented with 10 ng/ml of TGFβ2), and the support was removed. The resulting free-floating tissue was incubated for 48 hours or more. The resulting produced artificial skin model of the present disclosure had a multilayer structure consisting of an epithelial layer (epidermis), a basement membrane, and a stromal layer (dermis). The basement membrane and the stromal layer are characterized by being bound to each other by TG2, a bioadhesive.

### Example 2. Establishment of Conditions for Incubation of 3D Artificial Skin Model

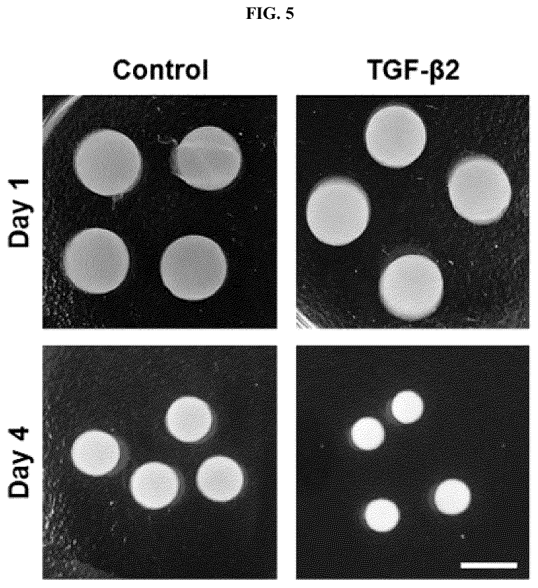

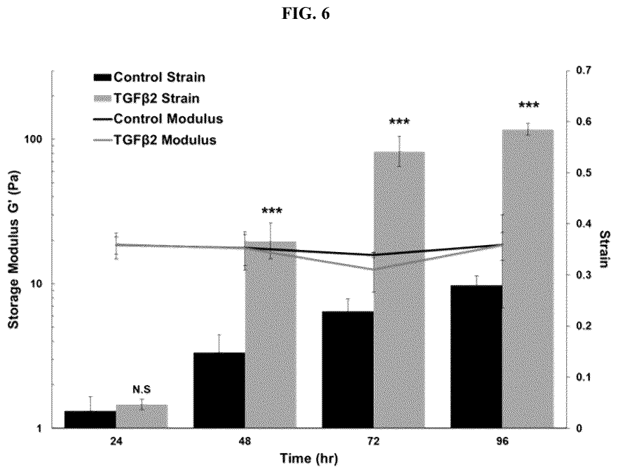

In order to form wavy ridges in the stromal layer corresponding to the dermal layer of normal skin tissue, tissue contraction is required at the stromal layer boundary that is in contact with the basement membrane. In order to confirm whether the artificial skin model of the present disclosure can mimic this function, a basement membrane+stromal layer model without epithelial cells (Example 1-4) was produced, incubated under conditions of 37° C. and 5% CO₂ for 96 hours. Incubation was performed using DMEM containing 10% FBS (fetal bovine serum) and 1% P/S (penicillin/streptomycin), and 10 ng/ml of TGF-β2 (transforming growth factor-beta 2) was additionally added to the test group. FIGS. 5 and 6, respectively, show the results of imaging the incubated model and the results of quantifying the image size relative to the control strain (0) based on 6 mm. The physical property was measured with Anton Paar MCR 92. As a result of the test, it was found that when TGF-β2 was added to the medium, tissue contraction was well induced at the boundary of the stromal layer. This is believed to be because TGF-β2 increased contraction by activating stromal cells (fibroblasts) in the stromal layer. Therefore, in the following study, a medium supplemented with 10 ng/ml of TGF-β2 was used.

Additionally, the basement membrane+stromal layer model without epithelial cells (Example 1-4) was produced and incubated for 24 hours under conditions of 37° C. and 5% CO₂. The model was divided into three groups and incubated under the conditions shown in Table 1 below.

TABLE 1

| Group | Culture conditions |
|---|---|
| Control | 24 hours of incubation with medium |
| | containing DMEM + 10% FBS + 1% |
| | P/S + 10 ng/ml TGF-β2 |
| Bleb after | 12 hours of incubation with control medium, |
| 12 hours | followed by 12 hours of additional |
| | incubation with medium supplemented |
| | with 10 μM blebbistatin |
| Bleb | 24 hours of incubation with control medium |
| | supplemented with 10 μM blebbistatin |

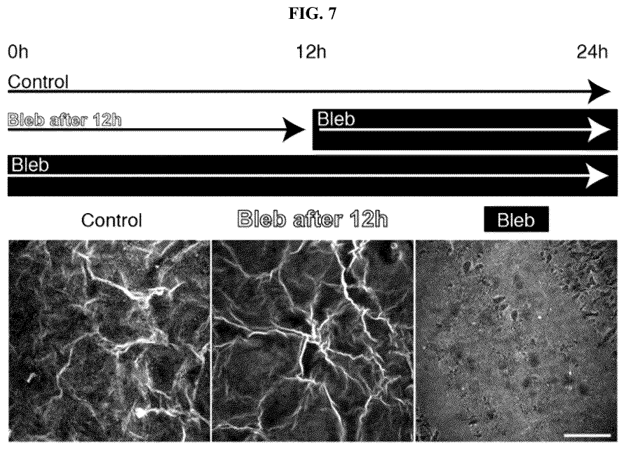

After incubation, each sample was imaged with a confocal fluorescence microscope at a wavelength of 488 nm, and the images were synthesized into two-dimensional images by max intensity projection using the ImageJ imaging program. The results are shown in FIG. 7. The test results showed that when the basement membrane+stromal layer artificial skin model was incubated in the medium containing blebbistatin from the beginning (=before tissue contraction), tissue contraction at the stromal layer boundary was inhibited. However, it could be seen that, in the sample in which tissue contraction had already occurred, tissue contraction was maintained even after subsequent treatment with blebbistatin.

### Example 3. Verification of Efficacy of 3D Artificial Skin Model

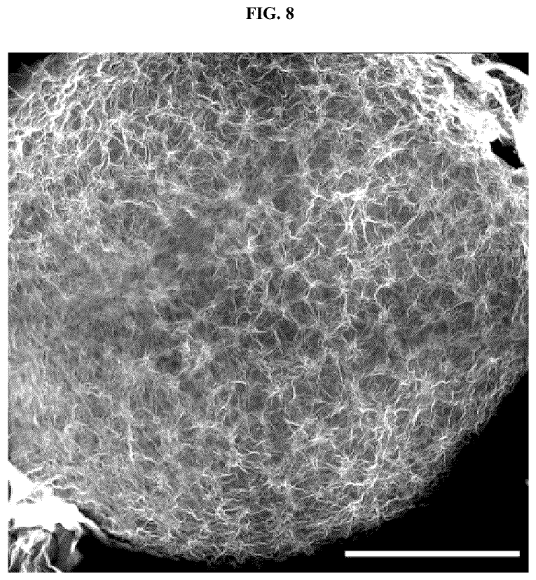

In order to confirm whether the three-dimensional (3D) artificial skin of the present disclosure well mimics the shapes of the ridges at the dermal-epidermal junction (DEJ), an epithelial layer+basement membrane+stromal layer model (Example 1-5) with both epithelial cells and stromal cells was produced and incubated with a medium having a composition of DMEM+10% FBS+1% P/S+10 ng/ml TGF-β2 for 24 hours under conditions of 37° C. and 5% CO₂. As a result of imaging the incubated model, it could be seen that the 3D artificial skin model of the present disclosure well mimicked the shape of the wavy ridges. The result is shown in FIG. 8.

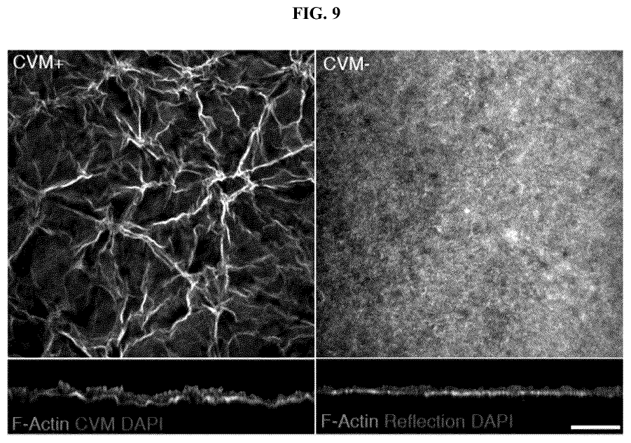

In addition, in order to evaluate the effect of the presence of the basement membrane in the 3D artificial skin model of the present disclosure, an epithelial layer+basement membrane+stromal layer model (CVM+) and an epithelial layer+stromal layer model (CVM−) were produced and cultured under the same conditions as described above. Each sample was fixed with 3% paraformaldehyde (PFA) for 15 minutes, washed with DPBS, and then stained with phalloidin and DAPI at room temperature. As a result of imaging the stained samples with a confocal fluorescence microscope at wavelengths of 305, 488, and 546 nm, it could be seen that the artificial skin model without the basement membrane failed to mimic the shape of the ridges at the DEJ, suggesting the importance of the basement membrane in the artificial skin model. The results are shown in FIG. 9.

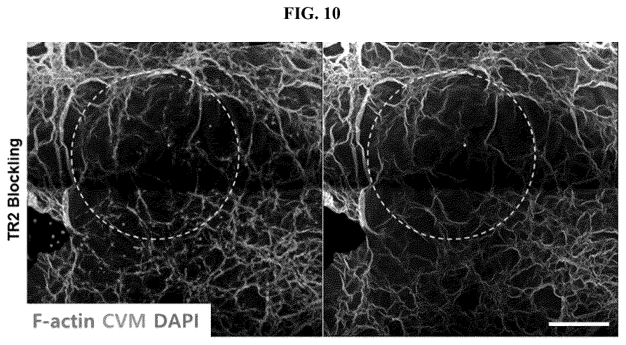

Additionally, when producing the 3D artificial skin model of the present disclosure, the base membrane was partially uncoated with TR2 in the step of producing the basement membrane+stromal layer model (Example 1-4) in order to evaluate the effect of coating of the basement membrane with TR2. Incubation and fluorescent staining of the artificial skin model were performed in the same manner as those in the test for the effect of the presence of the basement membrane, and the results of imaging the stained artificial skin model are shown in FIG. 10. As a result of the test, it was found that the TR2-uncoated portion (yellow circle in FIG. 10) showed decreased actin (F-actin) expression, suggesting that coating of the basement membrane with TR2 acts as an important factor in mimicking the shape of the ridges at the DEJ.

Although the present disclosure has been described in detail with reference to the specific features, it will be apparent to those skilled in the art that this description is only of a preferred embodiment thereof, and does not limit the scope of the present disclosure. Thus, the substantial scope of the present disclosure will be defined by the appended claims and equivalents thereto.

What is claimed is:

1. A method for producing a multilayer-structured artificial skin, the method comprising steps of:
(a) preparing a first layer including collagen and stromal cells; and
(b) preparing a second layer comprising cellulose acetate and collagen,
wherein the second layer is located on the first layer.

2. The method according to claim 1, wherein the first layer and the second layer are attached to each other by a bioadhesive.

3. The method according to claim 2, wherein the bioadhesive is transglutaminase 2 (TR2).

4. The method according to claim 1, wherein the artificial skin has ridges formed at a junction between the first layer and the second layer.

5. The method according to claim 1, wherein the stromal cells in step (a) are any one or more types of cells selected from the group consisting of fibroblasts, chondroblasts, osteoblasts, neuroglial cells, adipocytes, macrophages, and plasma cells.

6. The method according to claim 1, further comprising step (c) of preparing a third layer comprising epithelial cells.

7. The method according to claim 6, wherein the third layer is located on the second layer.

8. The method according to claim 6, wherein the epithelial cells in step (c) are any one or more types of cells selected from the group consisting of simple squamous epithelium, simple cuboidal epithelium, simple columnar epithelium, stratified squamous epithelium, stratified cuboidal epithelium, stratified columnar epithelium, pseudostratified epithelium, transitional epithelium, and glandular epithelium cells.

9. The method according to claim 1, further comprising step (d) of incubating the first layer and the second layer in a medium containing transforming growth factor-beta 2 (TGF-β2).

10. A multilayer-structured artificial skin comprising:
a first layer comprising collagen and stromal cells; and
a second layer comprising cellulose acetate and collagen,
wherein the second layer is located on the first layer.

11. The artificial skin according to claim 10, wherein the first layer and the second layer are attached to each other by a bioadhesive.

12. The artificial skin according to claim 10, wherein the artificial skin has ridges formed at a junction between the first layer and the second layer.

13. The artificial skin according to claim 10, wherein the stromal cells are any one or more types of cells selected from the group consisting of fibroblasts, chondroblasts, osteoblasts, neuroglial cells, adipocytes, macrophages, and plasma cells.

14. The artificial skin according to claim 10, further comprising a third layer comprising epithelial cells.

15. The artificial skin according to claim 14, wherein the epithelial cells are any one or more types of cells selected from the group consisting of simple squamous epithelium, simple cuboidal epithelium, simple columnar epithelium, stratified squamous epithelium, stratified cuboidal epithelium, stratified columnar epithelium, pseudostratified epithelium, transitional epithelium, and glandular epithelium cells.

16. The artificial skin according to claim 10, which is for use in skin aging research.

17. A method for screening a candidate substance for inhibiting skin aging, the method comprising steps of:
(a) preparing the artificial skin of claim 10;
(b) treating the artificial skin with a candidate substance for inhibiting skin aging; and
(c) checking changes in ridges at a junction between the first and second layers in the artificial skin.

18. The method according to claim 17, wherein, if a density of the ridges in step (c) increased, the candidate substance is determined to be effective in inhibiting skin aging.
